(19) United States
(12) Patent Application Publication
(10) Pub. No.: US 2022/0264953 A1
(43) Pub. Date: Aug. 25, 2022

(54) SUBSTANCE DELIVERY APPARATUS, SYSTEMS AND METHODS

(71) Applicant: Maury D. COLE, San Diego, CA (US)

(72) Inventor: Maury D. COLE, San Diego, CA (US)

(21) Appl. No.: 17/677,831

(22) Filed: Feb. 22, 2022

Related U.S. Application Data
(60) Provisional application No. 63/152,724, filed on Feb. 23, 2021.

(51) Int. Cl. A24F 40/46; A24F 40/42; A24F 40/30; A24F 40/60

(52) U.S. Cl. CPC A24F 40/46; A24F 40/42; A24F 40/30; A24F 40/60

(57) ABSTRACT

Substance supply apparatus, systems and methods are provided herein. In some aspects, a substance supply apparatus comprises a tank assembly coupled to a manifold. The tank assembly can comprise a cavity housing one or more electrical components, a set of heating elements electrically coupled to the electrical components, and wherein each heating element is configured to couple with a cartridge configured to house a substance to be heated. The manifold can comprise a plurality of delivery ports, wherein each delivery port is configured to couple with a cartridge that is also coupled to the heating element. In some aspects, a system comprises one or more supply apparatus, as well as one or more inhalation chambers, actuator devices, supply paths from the apparatus to an inlet port of the chambers, and one or more controllers.

### CROSS-REFERENCE TO RELATED APPLICATIONS

This application claims priority to U.S. Provisional Patent App. No. 63/152,724 filed on Feb. 23, 2021. This and all other extrinsic materials discussed herein, including publications, patent applications, and patents, are incorporated by reference in their entirety. Where a definition or use of a term in an incorporated reference is inconsistent or contrary to the definition of that term provided herein, the definition of that term provided herein applies and the definition of the term in the reference does not apply.

### BACKGROUND

### Field of the Invention

The embodiments described herein are generally directed to apparatus, systems and methods for substance delivery.

### Description of the Related Art

The destructive effects of alcohol and drugs such as THC (tetrahydrocannabinol), cocaine and other stimulants are vast and well known and have led to the creation of the National Institute on Drug Abuse (NIH) and National Institute of Alcohol and Alcohol Abuse (NIAAA). Over the years, investigators funded by NIH and NIAAA have developed several systems for the delivery of drugs and alcohol which include presenting the drug as a liquid diet; injecting alcohol via gastric, intraperitoneal or intravenous routes; and by exposing test subjects, e.g., rodents, to drug and alcohol vapors. Each method has advantages and drawbacks that are abundantly discussed in the available literature. However, one significant problem that has consistently plagued research is the difficulty in reaching predictable blood levels (BLs) of drug and alcohol and, if desired, maintaining them within given values. Injections of a specific concentration of drugs or alcohol can be used but this procedure is very stressful unless the animals are equipped with indwelling permanent cannula. This requires skillful surgical training. Gavage via the forceful placement of a special tube into the esophagus can be done but is also very stressful, particularly if it is used repeatedly. Also, both methods (injections and gavage) induce rapid peak BLs followed by the disappearance of drugs and/or alcohol from the circulation. They cannot be used to maintain constant BLs over time. Feeding drugs or alcohol in the diet presents the advantage of voluntary drug intake but does not provide the investigator with significant control over the amounts of drugs or alcohol consumed, and consequently BLs are not consistent.

All of these problems have been critical barriers in drug and alcohol research. For this reason, some have turned to delivery of drugs and alcohol through vapors, for example, through systems and methods described in U.S. Pat. No. 7,252,050 and U.S. patent application Ser. No. 15/954,400 to Cole.

### SUMMARY

The apparatus, systems and methods disclosed herein provide a drug supply/delivery system that can provide multiple drug delivery and testing options to one or more inhalation chambers. In some aspects, options for selective drug delivery to chambers from a single, passive drug delivery system, for delivery of a plurality of different drugs, a plurality of different amounts, and/or a plurality of different concentrations to at least one chamber from a single drug supply/delivery apparatus are provided so that different drugs, amounts, and/or concentrations of delivered drugs can be tested. In some aspects, options for different, concentrations, and/or amounts of drug vapor delivery to the same chamber are provided, for example by a negative pressure or vacuum pressure system connected to a test chamber which creates suction, drawing an inhalable drug through the inhalation test chamber for inhalation by a test animal (similar to an e-vape inhalation system similar to e-cigarettes).

The drug supply/delivery systems can comprise an apparatus housing a plurality of distinct drugs, each of which can be independently and separately heated (e.g., burned) to produce an inhalable drug, corresponding to each heated drug, for inhalation by the test animal. Each housed drug may differ in type, amount, and/or concentration. The system can be used in a passive mode of administration with each inhalable drug is delivered in an operator determined order and at operator determined time periods, or in a self-administration mode in which one of the inhalable drugs is delivered when an animal engages a nose poke or similar actuator and different ones of the inhalable drugs are delivered with each engagement of the nose poke or similar actuator. The system can be attached to one or more animal test chambers which allow for whole body exposure or a nose exposure restraining chamber.

In some aspects, a drug supply/delivery system as described herein can be attached or otherwise coupled to at least one substance inhalation chamber. The drug supply/delivery system can include a plurality of cartridges coupled to respective ones of a plurality of delivery ports or tubes, each capable of delivering inhalable drugs to the substance inhalation chamber under control of the controller. The drug supply/delivery system can include an apparatus having a plurality of heating elements, each arranged to apply heat to a respective cartridge so to heat (e.g., burn) a drug housed therein. The resulting inhalable drug can then be provided to the inhalation chamber under suction. A different drug, different amount of drug, and/or different concentration of drug may be heated (e.g., to a burn, or heating to a point but not burning) at each delivery port and supplied to the substance inhalation chamber. This allows experiments to be conducted with different drugs, amounts, and concentrations dependent on which cartridge is used to supply inhalable drug to its respective delivery port.

In an example implementation, a drug supply/delivery system allows users to place different amounts of a drug to be tested into different cartridges. When heated (e.g., burned), the contents of each cartridge can provide a “puff” or “drag” of inhalable drug at user-defined intervals and lengths as programed into the controller (e.g., by an operator) or in response to an animal engaging a nose poke or similar actuator in self-administration mode. Similarly, different types of drugs and/or concentrations maybe be housed in each cartridge.

The systems contemplated herein can provide more flexibility for the researcher to perform different experiments or drug testing using one or more different drugs, amounts, and/or concentrations of potentially harmful substances such as nicotine, cocaine, THC and other stimulants. This system also provides more flexibility for the researcher to perform different experiments or drug testing using one or more different amounts of drugs. A controller can be connected to various components such as heaters, pumps, mixing chambers, e-cigarette attachments, and flow meters of the system and store accumulated test data.

Any of the methods above may be embodied, individually or in any combination, in executable software modules of a processor-based system, such as a server, and/or in executable instructions stored in a non-transitory computer-readable medium.

Other features and advantages of various embodiments will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 illustrates a substance delivery apparatus, according to an embodiment;

FIGS. 2A-2B illustrate a manifold of a substance delivery apparatus, according to another embodiment;

FIG. 3 illustrates a base component of a substance delivery apparatus, according to another embodiment;

FIG. 4 illustrates a top plate of a substance delivery apparatus without heating elements, according to another embodiment;

FIG. 5 illustrates a substance delivery apparatus, according to another embodiment;

FIG. 6 illustrates a substance delivery apparatus, according to yet another embodiment;

FIG. 7 illustrates an example processing system, by which one or more of the processes described herein, may be executed, according to an embodiment; and

FIG. 8 illustrates a substance delivery system, according to an embodiment.

### DETAILED DESCRIPTION

The detailed description set forth below, in connection with the accompanying drawings, is intended as a description of various embodiments and is not intended to represent the only embodiments in which the disclosure may be practiced. The detailed description includes specific details for the purpose of providing a thorough understanding of the embodiments. However, it will be apparent that those skilled in the art will be able to understand the disclosure without these specific details. In some instances, well-known structures and components are shown in simplified form for brevity of description. Some of the surfaces have been left out or exaggerated for clarity and ease of explanation.

Certain embodiments as disclosed herein provide for a drug supply/delivery system (also referred to herein as a drug supply system, substance supply system, drug delivery system, substance delivery system, supply system, substance inhalation system, delivery system, and/or system) that provide for selective drug delivery from a substance supply/delivery apparatus (also referred to herein as a drug supply apparatus, substance supply apparatus, drug delivery apparatus, substance delivery apparatus, supply apparatus, delivery apparatus, and/or apparatus) comprising a plurality of drug delivery assemblies. In some embodiments, the supply system may be used in connection with any suitable inhalation chamber apparatus, including, for example, those described in U.S. Patent Publication No. 2018/0228990. For example, embodiments herein may be connected to one or more of the animal test chambers of the inhalation apparatus described in U.S. Patent Publication No. 2018/0228990. As an example, the supply system according to embodiments herein may be controlled by a controller to supply inhalable drug through a supply hose connected to an inlet of the inhalation chamber and directly into the inhalation chamber as described in U.S. Patent Publication No. 2018/0228990. The inhalable drug can mix with ambient air which can be obtained through an inlet opening of the supply system.

In some aspects, a substance supply apparatus is provided, comprising: a base component comprising a plurality of walls and a cavity configured to house one or more electrical components (e.g., wires, switches, bulbs, microprocessors); a top plate affixed to the base component, the top plate enclosing the cavity and comprising a plurality of heating elements electrically coupled to the electrical components, wherein the plurality of heating elements includes a first heating element configured to receive a first end of a cartridge configured to house a substance to be heated; and a manifold comprising a plurality of delivery ports including a first delivery port, and wherein the first delivery port is configured to couple with a second end of the cartridge and receive an inhalable drug produced in response to a heating of the substance by the first heating element.

In some aspects, the plurality of heating elements further comprises a second heating element configured to receive a first end of a second cartridge configured to house a second substance to be heated, and wherein the plurality of delivery ports comprises a second delivery port configured to couple with a second end of the second cartridge and receive a second inhalable drug produced in response to a heating of the second substance by the second heating element.

In some aspects, the plurality of heating elements further comprises a third heating element configured to receive a first end of a third cartridge configured to house a third substance to be heated, and wherein the plurality of delivery ports comprises a third delivery port configured to couple with a second end of the third cartridge and receive a third inhalable drug produced in response to a heating of the third substance by the third heating element.

In some aspects, the plurality of heating elements further comprises a fourth heating element configured to receive a first end of a fourth cartridge configured to house a fourth substance to be heated, and wherein the plurality of delivery ports comprises a fourth delivery port configured to couple with a second end of the fourth cartridge and receive a fourth inhalable drug produced in response to a heating of the fourth substance by the fourth heating element.

In some aspects, the plurality of heating elements further comprises a fifth heating element configured to receive a first end of a fifth cartridge configured to house a fifth substance to be heated, and wherein the plurality of delivery ports comprises a fifth delivery port configured to couple with a second end of the fifth cartridge and receive a fifth inhalable drug produced in response to a heating of the fifth substance by the fifth heating element.

In some aspects, the plurality of heating elements further comprises a sixth heating element configured to receive a first end of a sixth cartridge configured to house a sixth substance to be heated, and wherein the plurality of delivery ports comprises a sixth delivery port configured to couple with a second end of the sixth cartridge and receive a sixth inhalable drug produced in response to a heating of the sixth substance by the sixth heating element.

In some aspects, the plurality of heating elements further comprises a seventh heating element configured to receive a first end of a seventh cartridge configured to house a seventh substance to be heated, and wherein the plurality of delivery ports comprises a seventh delivery port configured to couple with a second end of the seventh cartridge and receive a seventh inhalable drug produced in response to a heating of the seventh substance by the seventh heating element.

In some aspects, the plurality of heating elements further comprises an eighth heating element configured to receive a first end of an eighth cartridge configured to house an eighth substance to be heated, and wherein the plurality of delivery ports comprises an eighth delivery port configured to couple with a second end of the eighth cartridge and receive an eighth inhalable drug produced in response to a heating of the eighth substance by the eighth heating element.

In some aspects, the plurality of heating elements further comprises a ninth heating element configured to receive a first end of a ninth cartridge configured to house a ninth substance to be heated, and wherein the plurality of delivery ports comprises a ninth delivery port configured to couple with a second end of the ninth cartridge and receive a ninth inhalable drug produced in response to a heating of the ninth substance by the ninth heating element.

In some aspects, the plurality of heating elements further comprises a tenth heating element configured to receive a first end of a tenth cartridge configured to house a tenth substance to be heated, and wherein the plurality of delivery ports comprises a tenth delivery port configured to couple with a second end of the tenth cartridge and receive a tenth inhalable drug produced in response to a heating of the tenth substance by the tenth heating element.

It should be appreciated that any suitable number of heating elements, cartridges, substances, and delivery ports may be included in contemplated embodiments (e.g., 2, 3, 4, 5, 6, 7, 8, 9, 10, at least 2, at least 3, at least 4, at least 5, at least 8, at least 10, at least 12, at least 15 of any of heating elements, cartridges, substances and/or delivery ports). In some aspects, the plurality of heating elements comprises ten heating elements arranged in a two by five array. In some aspects, the top plate is circular, and the plurality of heating elements comprises five heating elements.

In some aspects, the supply apparatus can comprise a controller configured to drive each of the heating elements of the plurality of heating elements separately and independently in accordance with a predefined order. In some aspects, each of the cartridges (e.g., each of the first, second and third cartridges) houses a different amount of the same substance. In some aspects, each of the cartridges (e.g., each of the first, second and third cartridges) houses a different type of substance. In some aspects, each of the cartridges (e.g., each of the first, second and third cartridges) houses a different concentration of the same substance.

In some aspects, a substance inhalation system is provided, comprising: an inhalation chamber having an inlet port having a fitting configured for releasable connection to a supply hose; an actuator device; a supply apparatus for supplying an inhalable substance to the inlet port; a supply path comprising the supply hose between the supply apparatus and chamber inlet port; and a controller connected to the supply apparatus and actuator device.

In some aspects, the supply apparatus can comprise: a base component comprising a plurality of walls and a cavity configured to house one or more electrical components, a top plate affixed to the base component, the top plate enclosing the cavity and comprising a plurality of heating elements electrically coupled to the electrical components, wherein the plurality of heating elements includes a first heating element configured to receive a first end of a cartridge configured to house a substance to be heated, and a manifold comprising an outlet having a fitting for releasable connection to the supply hose, and a plurality of delivery ports including a first delivery port, wherein the first delivery port is configured to couple with a second end of the cartridge and receive an inhalable drug produced in response to a heating of the substance by the first heating element.

In some aspects, the supply apparatus can comprise any of the supply apparatuses described herein.

In some aspects, the controller can be configured to turn on one or more heating elements of the plurality of heating elements. In some aspects, the controller is configured for user selection of a self-administration mode or passive mode, whereby the controller sequentially and separately turns on each heating element of the plurality of heating elements on separate and distinct activations of the actuator device by a test animal in the chamber when in a self-administration mode, and separately turns on each heating element of the plurality of heating elements in accordance with a predetermined order when in a passive mode.

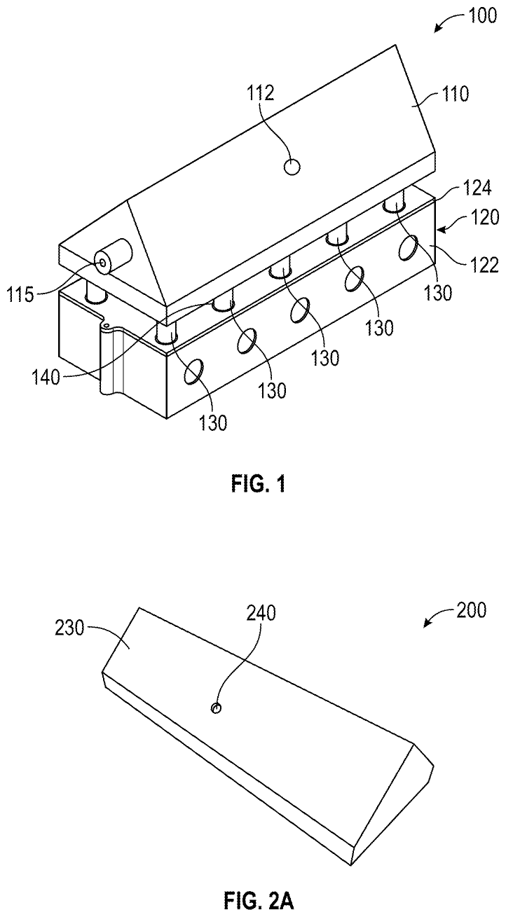

FIG. 1 illustrates an embodiment of a supply apparatus according to some embodiments disclosed herein. The supply apparatus 100 includes a manifold 110 and a tank assembly 120. The tank assembly 120 comprises a tank base 122 removably or fixedly coupled (e.g., removably attached) to a tank top plate 124 forming a cavity there between. In some aspects, tank base 122 and tank top plate 124 can be coupled to one another via a hinge mechanism, and may include one or more fastening or locking mechanisms. In some aspects, tank base 122 and tank top plate 124 can be removably attached to one another via one or more fastening mechanisms (e.g., screws, nails, rivets, adhesive, snap and the like).

The manifold 110 comprises an inlet opening 112 configured to allow ambient air may enter the manifold and, for example, mix with an inhalable drug therein. Manifold 110 comprises an outlet 115, which can be releasably connected to a supply hose (not shown) via a quick connect/disconnect or other fitting between the end of the supply hose and the outlet 115.

Supply apparatus 100 further comprises a plurality of cartridges 130 or cylinders, each configured to house a drug to be tested. For example, each cartridge may house an amount plant-based drug, such as nicotine, cocaine, THC, and/or other stimulants. The type of drug housed in each cartridge of an apparatus or system may be the same or different, the amount housed therein may be varied amongst the plurality of cartridges, and/or the concentration may be varied. In an example implementation, a varied amount of a cigarette or different cigarettes maybe be housed in each cartridge (e.g., an operator may divide a cigarette into portions that are representative of a “puff” or “drag” of the cigarette) for testing effects on a test animal of less than the entirety of the cigarette(s). Each cartridge may be connected to a heating element 140, for example, via a quick connect/disconnect fitting between the cartridge and the heating element. A mouthpiece may be inserted to an upper end of one or more cartridges and a portion of a filter may be placed therein. Manifold 110 can comprise a chamber having a set of delivery ports or openings configured to allow passage of one or more inhalable drugs from one or more cartridges into the manifold.

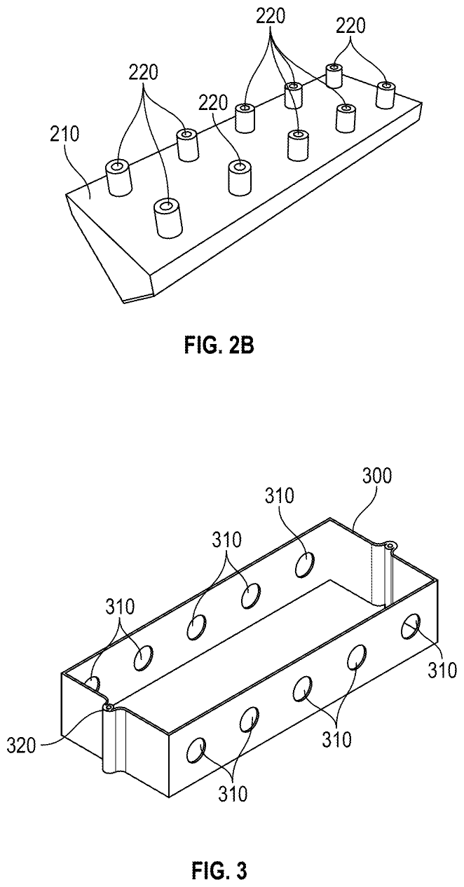

FIGS. 2A-2B illustrate a manifold 200 according to some embodiments disclosed herein. As shown in FIGS. 2A-2B, the manifold can comprise a chamber having a set of delivery ports that each comprises an opening configured for passage of an inhalable drug from within a cartridge into the manifold. Here, manifold comprises a chamber having a base 210 including a plurality of delivery ports 220, and a cover 230 having one or more inlet openings 240. FIG. 2B illustrates a plurality of delivery ports that connect to the plurality of cartridges. Each cartridge, delivery port, and heating element may be components that make up an individual drug delivery assembly, such that the supply system and/or supply apparatus comprises a plurality of drug delivery assemblies. When a mouthpiece is attached to the upper end of the cartridge, the respective delivery port may be arranged to receive the mouthpiece via quick connect/disconnect or other fitting between the delivery port and the mouthpiece. The mouthpiece may house a portion of a filter, so that when the drug is burned, smoke is supplied to the manifold via the mouthpiece thereby mimicking a “puff” or “drag” of the contents in the cartridge.

While the manifold 200 shown in FIGS. 2A-2B has a generally rectangular base with a cover having four side walls and an A-frame ceiling, it should be appreciated that manifolds having any suitable shape and size are contemplated. For example, a manifold can comprise a flask or other device having an inlet for ambient air and an outlet fluidly coupled to a chamber, and a plurality of delivery arms or tubes extending between a plurality of delivery ports (or openings of the flask) and a plurality of cartridges.

FIG. 3 illustrates a tank base according to some embodiments disclosed herein.

Tank base 300 comprises one or more side walls, a bottom wall, and an open top. A cavity, compartment or recess is formed by the side walls and bottom wall. As shown in FIG. 3, two of the side walls comprise a plurality of openings. However, it will be appreciated that any of the side walls may comprise one or more openings, the opening need not be limited to only certain side walls. In some aspects, the cavity may house a controller (not shown) or electrical components coupled to an external controller. In some aspects, the plurality of openings can be configured to receive electrical components for driving the heating elements. In some aspects, the plurality of openings can be configured and arranged to receive an electrical lead via a quick connect/disconnect or other fitting. Each respective electrical lead may supply power to each respective heating element. In some aspects, each electrical lead can be connected to a controller via a wired or wireless connection.

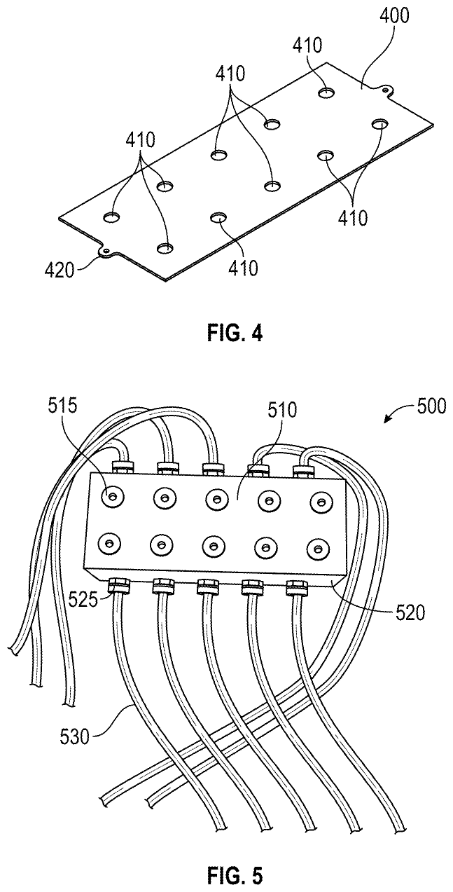

FIG. 4 illustrates a schematic drawing of the tank top plate 400 according to some embodiments disclosed herein. The tank top plate 400 comprises a plurality of openings 410 for receiving a set of heating elements, as described herein. A tank base (e.g., 300) and tank top plate (e.g., 400) can be coupled, attached, and/or fastened to one another in any suitable manner. For example. A tank base can comprise a fastener receiver 320, a tank top plate can comprise a complementary fastener receiver 420 that aligns with fastener receiver 320, and a fastener (e.g., a screw) can be fastened through fastener receiver 420 and at least a portion of fastener receiver 320.

While the disclosure herein is generally directed to tanks having a base and a top plate, it should be appreciated that in some aspects, a tank can comprise any suitable housing that houses electrical components, and includes a set of heating elements/heat producing objects (e.g., ceramic, metal (e.g., aluminum) or glass heating elements) configured to couple to a set of cartridges for housing a substance that can be heated to produce an inhalable substance. In some aspects, the heating elements are further configured to couple to a set of connectors for providing electrical or other power to the heating elements. For example, a tank can comprise a base plate and a top component that includes an upper wall including a plurality of openings for receiving a set of heating elements, and a set of side walls including a plurality of openings for one or more of receiving electrical components and receiving a fitting and/or an electrical lead via the fitting.

FIGS. 1-4 illustrate example supply apparatuses comprising a plurality of drug delivery assemblies having a plurality of cartridges for housing separate drugs to be tested. The drug housed therein may be burned via each respective heating element, similar to drug delivery systems described in U.S. Patent Application Publication No. 2018/0228990. The cartridges may be removably mounted by, for example, threaded engagement with a respective heating element disposed within a respective opening of the top plate. Each replaceable cartridge may contain a drug to be tested, such as nicotine or other plant-based drugs, which creates an inhalable drug (e.g., drug infused smoke) when heated (e.g., burned or heated to a point below burning) via the heating element. The cartridges can be of more robust construction then e-cigarette cartridges. Each cartridge may be pre-filled with a desired drug, amount of drug, and/or concentration or drug. In some aspects, the drug may be injected through an open top of the cartridge. After filling, a quick connect/disconnect fitting may be attached to the top for releasable connection to a delivery port of the manifold. In some embodiments, for example where a mouthpiece is attached to a cartridge, the delivery port may be releasably connected to the mouthpiece. The tank base may also contain the power supply source (e.g., a battery) for the heating elements and/or a controller, which may be connected to a control box via wired or wireless connections. In some embodiments, the power supply and/or controller may be external to the tank base.

Each cartridge outlet can be connected to a respective delivery port via quick connect/disconnect or other fitting to provide drug infused smoke, for example, via suction, into the manifold and to the outlet. The outlet can be connected to a chamber via a supply hose and the chamber can be connected to a vacuum source, as described in U.S. Patent Application Publication No. 2018/0228990. In some aspects, when supply apparatus is turned off, ambient air only is supplied to the manifold via inlet opening.

FIG. 5 illustrates a supply apparatus 500 in accordance with some embodiments herein, except that the manifold is removed for illustrative purposes. As described herein, the tank top plate 510 comprises a plurality of heating element 515 and is affixed to the base 520. The base may comprise mounting flanges (not shown). Each heating element may include a 510 thread screw or other adaptor, which can make an electronic connection to a cartridge. In some embodiments, a vape pen or other device having a battery or cigarette attachment device may also be attached to the thread screw adaptor. Each opening of the tank base can be arranged to receive an electrical lead 530 via a quick connect/disconnect 525. Each respective electrical lead may supply power to each respective heating element and be connected to the controller via a wired or wireless connection. In various embodiments, a single controller can be provided for controlling the heating elements according to a test algorithm in passive mode and/or in self-administration mode. While the example shown in FIG. 5 illustrates the electrical leads connected along the long side wall of the base, the present disclosure is not so limited. For example, one or more electrical leads may be connected to any side walls, either the shorter side wall or longer one, or any other portion of the tank.

In the example implementations shown in FIGS. 1-5, ten heating elements are provided for receiving ten cartridges, some or all of which can house different drug contents. Contents in each cartridge may differ in type of drug, amount of drug, and/or drug concentration. While some examples herein provide for ten heating elements in a M by N array (where M equals two and N equals five), this disclosure is not so limited. Supply systems within the scope of this disclosure comprise any number of two or more heating elements (and thus any number of two or more drug delivery assemblies) arranged in any suitable manner. For example, in some aspects, the heating elements can be arranged in any N×B array, where N is an integer ranging from 1 or more and M is an integer ranging from 2 or more. Alternatively, in some aspects, M may be an integer of 1 or more and N ranges from 2 or more. In some other aspects, the heating elements can be arranged in a circular shape, polygonal shape (e.g., triangular, hexagonal, pentagonal) and/or an irregular shape along one or more surfaces of a tank. In some aspects, the heating elements can be positioned along openings of any portion(s) or component(s) of a tank. In some aspects, the heating elements can be positioned along openings of a tank top plate. In some aspects, the heating elements can be positioned along openings of a tank base. In some aspects, the heating elements can be positioned along openings of a tank base and a tank top plate.

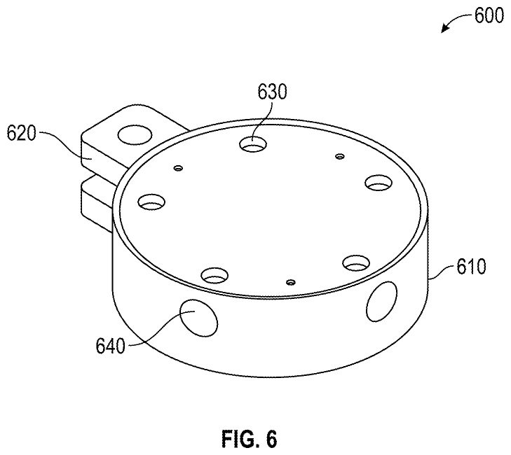

Now turning to FIG. 6, a supply apparatus 600 according to another embodiment is provided (without a manifold or cartridges). Supply apparatus 600 comprises a tank base 610 coupled to a tank top plate 620 via one or more fasteners. Each of tank base 610 and tank top plate 620 comprises a portion of a fastener assembly (here, flanges having fastener receiving openings sized and dimensioned to receive a fastener screw or portion thereof) to fasten tank base 610 with tank top plate 620. Tank top plate 620 comprises a set of heating elements 630 (five as shown), which can each be positioned in an opening of tank top plate 620. The heating elements can be configured to each receive a first end of a cartridge configured to house a substance to be heated to form an inhalable drug. A manifold (not shown) can comprise an outlet having a fitting for releasable connection to a supply hose, and a plurality of delivery ports each configured to couple with a second end of a cartridge and receive an inhalable drug produced in response to a heating of the substance by the first heating element. Tank base 610 comprises a side wall with a set of openings 640. In some aspects, a cavity is provided within the tank (e.g., within side wall(s), bottom wall, and top plate), which may house a controller (not shown), other electrical components, or electrical components coupled to an external controller. In some aspects, the plurality of openings 640 can be configured to receive electrical components for driving the heating elements. In some aspects, the plurality of openings can be configured and arranged to receive an electrical lead via a quick connect/disconnect or other fitting. Each respective electrical lead may supply power to each respective heating element. In some aspects, each electrical lead can be connected to a controller via a wired or wireless connection.

Some embodiments herein provide for a plant-based e-vape tank that enables a user to burn a plurality of different amounts of plant-based drug, a plurality of drug types, and/or a plurality of concentrations via respective drug delivery assembles. In an example, an operator is able to burn at least 5 grams, at least 6 grams, at least 7 grams, at least 8 grams, at least 9 grams, at least 10 grams, at least 11 grams, at least 12 grams, at least 13 grams, at least 14 grams, at least 15 grams, up to 15 grams, up to 14 grams, up to 13 grams, up to 12 grams, up to 11 grams, or up to 10 grams of a plant-based drug or portion of a cigarette in a single drug delivery assembly.

Embodiments herein may be controlled via a controller or computer system, such as those described in U.S. Pat. No. 7,252,050 and/or U.S. Patent Publication No. 2018/0228990, the contents of which are incorporated herein by reference and may incorporate standard software as provided by Med Associates Inc, Coulbourn Instruments, or La Jolla Alcohol Research, Inc. (LJARI) for controlling and monitoring components of the supply or delivery system and exhaust system, as well as for controlling timing and flow rates to connected test chambers, collecting test data from test subjects, and detecting nose poke actuation in test chamber when in the self-administration mode.

Advantageously, the apparatus, systems and methods disclosed herein give the user the ability to look at a number of behavioral studies in behavioral research, and allow an operator to compare the effects of different drugs, amounts, and concentrations through a single system. For example, a user can compare e-vape/e-liquid housed in one cartridge to plant-based THC or CBD or tobacco products in other cartridges. Some known disadvantages of some other systems include the inability to provide for self-administration of inhalable drugs from freshly burned plant-based drugs. Freshly burning a drug typically corresponded to burning a full cigarette or other plant-based drug instead of a “puff” or “drag”, thus failing to provide a real-life model. In real-life scenarios, a person smoking a cigarette “puffs” or takes a “drag” of a portion of an entire amount of drug (e.g., a portion of a cigarette). The apparatus, systems and methods disclosed herein are capable of mimicking such “puffs” or a “drag,” instead requiring burning an entire cigarette.

Thus, embodiments herein provide a supply system, whereby an operator is able to measure out various amounts of a drug (e.g., lengths of cigarette, cigar, a rolled drug, e-liquid), place each amount into a separate cartridge, and burn each portion separately under control of the controller, or, for example, a nose poke in self-administration mode. Each cartridge may also have different types of drugs and/or different concentrations and/or different amounts. In various embodiments, a filter can be disposed in a non-burning section of the supply system (for example a mouthpiece or elsewhere in the supply system outside of the cartridge away from the heat source) so to act upon smoke from each cartridge. In this way, a user can perform behavioral science on usage that mimics real-life situations and scenarios.

In an example implementation of some embodiments disclosed herein, if an average cigarette is 3.5 inches long, in a real-life scenario no one smokes the whole cigarette in one “drag”. Instead, a single “drag” may correspond to smoking a portion (e.g., between 1-10 mm, between 4-8 mm, between 1/12^{th} and 1/20^{th} a length of the cigarette, about 1/16^{th}) of the cigarette. Embodiments herein provide for testing effects of different portions of a single cigarette. An operator can measure out various lengths of a cigarette or different amounts of plant-based drugs and place each measured amount into a separate cartridge. A filter may be divided into separate pieces, each inserted into a mouthpiece attached to each cartridge. When the contents of a respective cartridge are burned, the resulting inhalable drug can mimic a single “drag” corresponding to the measured amount of the drug contained therein, thereby representing a real-life effect of a single “drag” of the drug.

In an example implementation, the supply system disclosed herein may be connected to a supply hose of an inhalation apparatus as described in, for example, U.S. Patent Publication No. 2018/0228990. Desired amounts, concentrations, combinations, or types of a drug or drugs to be tested may be placed in respective cartridges, and the cartridges can be connected or otherwise coupled to the manifold. A vacuum pump of the system (e.g., a vacuum pump coupled to the test chamber) can be turned on slowly until a flow meter or liter per minute gauge reaches the desired air flow level. The power of the controller can be turned to initiate a testing algorithm and power may be supplied to a first heating element according to the test algorithm. Drug contained in the first cartridge attached to the first heating element can be burned producing an inhalable drug, which enters the manifold. As described in U.S. Patent Publication No. 2018/0228990, the smoke in the manifold is provided to the inhalation apparatus (e.g., a test chamber) via the manifold outlet and supply hose due to a suction caused by the vacuum source.

At a time subsequent to driving the first heating element, the controller can supply power to a second heating element according to the test algorithm and repeat the process. The controller may separately drive each heating element based on the desired test protocol to supply inhalable drug corresponding to the differing amounts, concentrations, combinations, or types of a drug or drugs to the inhalation chamber. In this way, behavioral effects of different “puffs” or “drag” amounts can be tested. It should be appreciated that different amounts of a drug, different concentrations of a drug, different types of drugs, different variations of drugs, different combinations of drugs, and/or any other parameters can be tested using the apparatus, systems and methods disclosed herein.

In self-administration mode, in response to an animal acting on a nose poke or other actuator device, the controller can supply power to one or more of the heating elements according to a test algorithm. As described above, a corresponding amount of inhalable drug(s) can be supplied to the chamber. Then, when the animal acts on the nose poke or other actuator device again, a second heating element can be driven thereby suppling a different amount, type or concentration of drug to the chamber. It should be appreciated that contemplated systems can comprise one or more chambers configured to house an animal, and one or more nose pokes or other actuator devices in one or more of the chambers. One nose poke or actuator device can be associated with one, some or all of the drug delivery assemblies of a drug delivery system. In some aspects, one nose poke or other actuator device is associated with each of the drug delivery assemblies of a system. In some aspects, one nose poke or other actuator device is associated with two or more of the delivery assemblies of a system (e.g., delivery assemblies housing lower concentrations of a drug; delivery assemblies housing higher concentrations of a drug; delivery assemblies housing a specific type of drug). In some aspects, a set of nose pokes or other actuator devices are provided, each corresponding to a delivery assembly of the set of delivery assemblies of a system.

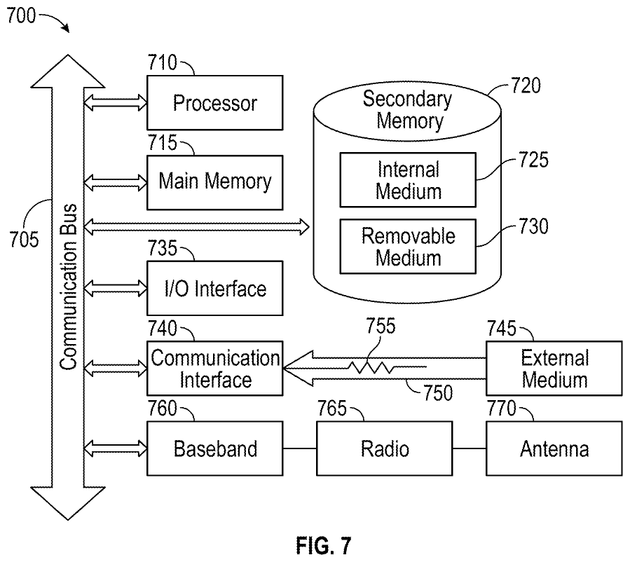

FIG. 7 is a block diagram illustrating an example wired or wireless system 700 that may be used in connection with various embodiments described herein. For example, the system 700 may be used as or in conjunction with the embodiments of the supply system disclosed herein, for example, as the controller described herein. The system 700 can be a conventional personal computer, computer server, personal digital assistant, smart phone, tablet computer, or any other processor enabled device that is capable of wired or wireless data communication. More particularly, the system 700 may represent the application server. Other computer systems and/or architectures may be also used, as will be clear to those skilled in the art.

The system 700 preferably includes one or more processors, such as processor 710. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 710.

In one embodiment, the controller can be configured with more than one processor in order to separately process the incoming signals from a plurality of sensing devices, while a yet further processor is responsible for handling communication with the gateway device. In another embodiment, the gateway device may also be configured with a plurality of processors, with one processor configured to receive and process data from the central control device, a second processor configured to generate graphical user interfaces to display the received data to the user on a display of the gateway device and a third processor to communicate with the remote server. For a portable electronic device such as the central control device and gateway device, the processors may be low power processors to reduce power consumption on the devices' batteries.

The processor 710 is preferably connected to a communication bus 705. The communication bus 705 may include a data channel for facilitating information transfer between storage and other peripheral components of the system 700. The communication bus 705 further may provide a set of signals used for communication with the processor 710, including a data bus, address bus, and control bus (not shown). The communication bus 710 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPM”), IEEE 696/S-100, and the like. These standards may be applicable to the remote server, while additional or varying standards may apply to portable electronic devices such as the central control device or sensing devices.

System 700 preferably includes a main memory 715 and may also include a secondary memory 720. The main memory 715 provides storage of instructions and data for programs executing on the processor 710. The main memory 715 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 720 may optionally include an internal memory 725 and/or a removable medium 730, for example a floppy disk drive, a magnetic tape drive, a compact disc (“CD”) drive, a digital versatile disc (“DVD”) drive, etc. The removable medium 730 is read from and/or written to in a well-known manner. Removable storage medium 730 may be, for example, a floppy disk, magnetic tape, CD, DVD, SD card, etc.

The removable storage medium 730 is a non-transitory computer readable medium having stored thereon computer executable code (i.e., software) and/or data. The computer software or data stored on the removable storage medium 730 is read into the system 700 for execution by the processor 710.

In alternative embodiments, secondary memory 720 may include other similar means for allowing computer programs or other data or instructions to be loaded into the system 700. Such means may include, for example, an external storage medium 745 and an interface 740. Examples of external storage medium 745 may include an external hard disk drive or an external optical drive, or and external magneto-optical drive.

Other examples of secondary memory 720 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage media 730 and communication interface 740, which allow software and data to be transferred from an external medium 745 to the system 700.

System 700 may also include an input/output (“I/O”) interface 735. The I/O interface 735 facilitates input from and output to external devices. For example the I/O interface 735 may receive input from a keyboard or mouse and may provide output to a display. The I/O interface 735 is capable of facilitating input from and output to various alternative types of human interface and machine interface devices alike.

System 700 may also include a communication interface 740. The communication interface 740 allows software and data to be transferred between system 700 and external devices (e.g. printers), networks, or information sources. For example, computer software or executable code may be transferred to system 700 from a network server via communication interface 740. Examples of communication interface 740 include a modem, a network interface card (“NIC”), a wireless data card, a communications port, a PCMCIA slot and card, an infrared interface, and an IEEE 1394 fire-wire, just to name a few.

Communication interface 740 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 740 are generally in the form of electrical communication signals 755. These signals 755 are preferably provided to communication interface 740 via a communication channel 750. In one embodiment, the communication channel 750 may be a wired or wireless network, or any variety of other communication links. Communication channel 750 carries signals 755 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 715 and/or the secondary memory 720. Computer programs can also be received via communication interface 740 and stored in the main memory 715 and/or the secondary memory 720. Such computer programs, when executed, enable the system 700 to perform the various functions of the present invention as previously described.

In this description, the term “computer readable medium” is used to refer to any non-transitory computer readable storage media used to provide computer executable code (e.g., software and computer programs) to the system 700. Examples of these media include main memory 715, secondary memory 720 (including internal memory 725, removable medium 730, and external storage medium 745), and any peripheral device communicatively coupled with communication interface 740 (including a network information server or other network device). These non-transitory computer readable mediums are means for providing executable code, programming instructions, and software to the system 700.

In an embodiment that is implemented using software, the software may be stored on a computer readable medium and loaded into the system 700 by way of removable medium 730, I/O interface 735, or communication interface 740. In such an embodiment, the software is loaded into the system 700 in the form of electrical communication signals 755. The software, when executed by the processor 710, preferably causes the processor 710 to perform the inventive features and functions previously described herein.

The system 700 also includes optional wireless communication components that facilitate wireless communication over a voice and over a data network. The wireless communication components comprise an antenna system 770, a radio system 765 and a baseband system 760. In the system 700, radio frequency (“RF”) signals are transmitted and received over the air by the antenna system 770 under the management of the radio system 765.

In one embodiment, the antenna system 770 may comprise one or more antennae and one or more multiplexors (not shown) that perform a switching function to provide the antenna system 770 with transmit and receive signal paths. In the receive path, received RF signals can be coupled from a multiplexor to a low noise amplifier (not shown) that amplifies the received RF signal and sends the amplified signal to the radio system 765.

In alternative embodiments, the radio system 765 may comprise one or more radios that are configured to communicate over various frequencies. In one embodiment, the radio system 765 may combine a demodulator (not shown) and modulator (not shown) in one integrated circuit (“IC”). The demodulator and modulator can also be separate components. In the incoming path, the demodulator strips away the RF carrier signal leaving a baseband receive audio signal, which is sent from the radio system 765 to the baseband system 760.

If the received signal contains audio information, then baseband system 760 decodes the signal and converts it to an analog signal. Then the signal is amplified and sent to a speaker. The baseband system 760 also receives analog audio signals from a microphone. These analog audio signals are converted to digital signals and encoded by the baseband system 760. The baseband system 760 also codes the digital signals for transmission and generates a baseband transmit audio signal that is routed to the modulator portion of the radio system 765. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 770 where the signal is switched to the antenna port for transmission.

The baseband system 760 is also communicatively coupled with the processor 710. The central processing unit 710 has access to data storage areas 715 and 720. The central processing unit 710 is preferably configured to execute instructions (i.e., computer programs or software) that can be stored in the memory 715 or the secondary memory 720. Computer programs can also be received from the baseband processor 760 and stored in the data storage area 715 or in secondary memory 720, or executed upon receipt. Such computer programs, when executed, enable the system 700 to perform the various functions of the present invention as previously described. For example, data storage areas 715 may include various software modules (not shown) that are executable by processor 710.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

In some aspects, the drug delivery system can include or be interfaced with a processing system 700, e.g., via I/O interface 735, and processing system 700 can be a controller that is configured to control the supply of an inhalable drug to an inhalation chamber of a drug delivery system. In some aspects, the processing system 700 can cause one or more heating elements of a system to heat a drug contained in a cartridge coupled thereto, for example, in accordance with a test algorithm stored in, for example, main memory 715, and/or separately and independently in accordance with a predefined order. In some aspects, processing system 700 can monitor components of the drug delivery system, control timing and flow rates to connected test chambers, collect test data, detect nose poke or other actuation in a test chamber, and store accumulated data.

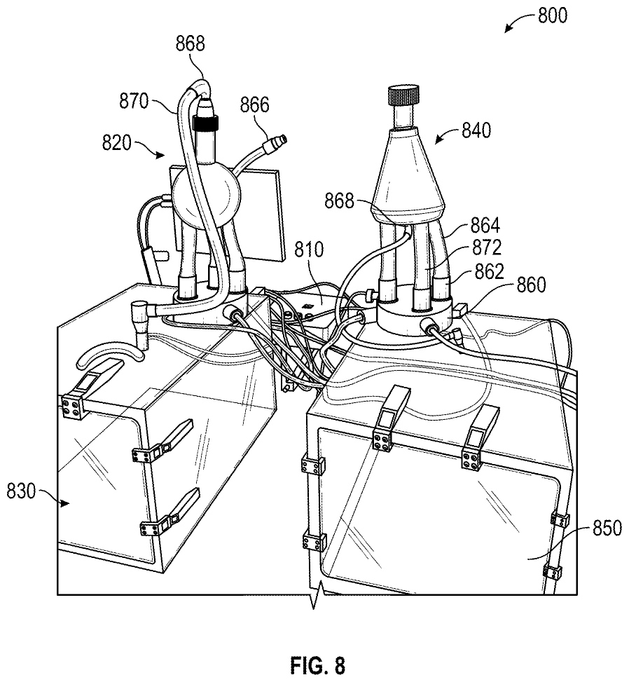

FIG. 8 illustrates a system of the inventive subject matter comprising a controller 810, a first drug delivery apparatus 820 fluidly coupled to controller 810 and first test chamber 830, and a second drug delivery apparatus 840 fluidly coupled to controller 810 and second test chamber 850. Each drug delivery apparatus (820, 850) comprises a tank assembly 860, a set of cartridges 862 (five shown on each apparatus), a manifold 864, which can comprise an inlet 866 for ambient air, an outlet 868 that fluidly couples with a test chamber via tube 870, and a set of openings to receive an inhalable drug via a set of connectors (e.g., tubes) 872 directly or indirectly coupled to the cartridges 862.

In some aspects, a tank assembly can comprise a tank base removably or fixedly coupled (e.g., removably attached) to a tank top plate forming a cavity there between. In some aspects, a tank base and a tank top plate can be coupled to one another via a hinge mechanism, and may include one or more fastening or locking mechanisms. In some aspects, a tank base and tank top plate can be removably attached to one another via one or more fastening mechanisms (e.g., screws, nails, rivets, adhesive, snap and the like). In some aspects, the cavity may house a controller (not shown) or electrical components coupled to an external controller. In some aspects, a plurality of openings can be configured to receive electrical components for driving the heating elements. In some aspects, a plurality of openings can be configured and arranged to receive an electrical lead via a quick connect/disconnect or other fitting. Each respective electrical lead may supply power to a respective heating element. In some aspects, each electrical lead can be connected to a controller via a wired or wireless connection. The tank, for example on a top plate, can comprise a plurality of heating elements. For example, a top plate can comprise a plurality of openings for receiving a set of heating elements.

The cartridges 862 or cylinders can each be configured to house a drug to be tested. For example, each cartridge may house an amount plant-based drug, such as nicotine, cocaine, THC, and/or other stimulants. The type of drug housed in each cartridge of an apparatus or system may be the same or different, the amount housed therein may be varied amongst the plurality of cartridges, and/or the concentration may be varied. In an example implementation, a varied amount of a cigarette or different cigarettes maybe be housed in each cartridge (e.g., an operator may divide a cigarette into portions that are representative of a “puff” or “drag” of the cigarette) for testing effects on a test animal of less than the entirety of the cigarette(s). Each cartridge may be connected to a heating element of the tank assembly, for example, via a quick connect/disconnect fitting between the cartridge and the heating element. In some aspects, a mouthpiece may be inserted to an upper end of one or more cartridges and a portion of a filter may be placed therein.

In some aspects, manifold 864 can comprise a chamber (e.g., a flask) having a set of delivery ports or openings configured to allow passage of one or more inhalable drugs from one or more cartridges into the manifold. A manifold inlet 866 can be configured to allow ambient air to enter the manifold and, for example, mix with an inhalable drug therein. Outlet 868 of the manifold can be releasably connected to a supply hose or tube 870, for example, via a quick connect/disconnect or other fitting between the end of the tube 870 and the outlet 868. Each cartridge, delivery port, and heating element may be components that make up an individual drug delivery assembly. As shown, system 800 comprises two sets of five delivery assemblies. However, any suitable number of delivery assemblies may be included in a system with any suitable arrangement. When a mouthpiece is attached to the upper end or other portion of a cartridge, the respective delivery port may be arranged to receive the mouthpiece via quick connect/disconnect or other fitting between the delivery port and the mouthpiece. The mouthpiece may house a portion of a filter, so that when the drug is burned, smoke is supplied to the manifold via the mouthpiece thereby mimicking a “puff” or “drag” of the contents in the cartridge.

In some aspects, a controller 810 can comprise or be communicatively coupled to a user interface. In some aspects, a controller can supply power to a second heating element according to the test algorithm and repeat the process. The controller can be configured to separately drive each heating element based on the desired test protocol and/or user inputs via the user interface to supply inhalable drug corresponding to the differing amounts, concentrations, combinations, or types of a drug or drugs to the inhalation chamber. In this way, behavioral effects of different “puffs” or “drag” amounts can be tested. It should be appreciated that different amounts of a drug, different concentrations of a drug, different types of drugs, different variations of drugs, different combinations of drugs, and/or any other parameters can be tested using the apparatus, systems and methods disclosed herein.

In self-administration mode, in response to an animal acting on a nose poke or other actuator device, the controller can supply power to one or more of the heating elements, for example, in accordance with a test algorithm. As described above, a corresponding amount of inhalable drug(s) can be supplied to the test chamber. Then, when the animal acts on the nose poke or other actuator device again, a second heating element can be driven thereby suppling the same amount of the same drug, or a different amount, type or concentration of drug to the chamber. It should be appreciated that contemplated systems can comprise any suitable number of test chambers configured to house any suitable number of animals, and one or more nose pokes or other actuator devices can be provided in one or more of the chambers. In some aspects, one nose poke or actuator device can be associated with one, some or all of the drug delivery apparatus and/or assemblies of a drug delivery system. In some aspects, one nose poke or other actuator device is associated with only one of the drug delivery assemblies of a system, and multiple nose pokes can be provided, for example, to match the number of drug delivery assemblies. In some aspects, one nose poke or other actuator device is associated with two or more of the delivery assemblies of a system (e.g., delivery assemblies housing lower concentrations of a drug; delivery assemblies housing higher concentrations of a drug; delivery assemblies housing a specific type of drug). In some aspects, a set of nose pokes or other actuator devices are provided, each corresponding to a delivery assembly of the set of delivery assemblies of a system.

It should be appreciated that a controller can be communicatively coupled to any suitable number of test chambers and/or any suitable number of drug delivery apparatuses having any suitable number of drug delivery assemblies.

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

Thus, the claims are not intended to be limited to the aspects shown herein, but is to be accorded the full scope consistent with the language claims, wherein reference to an element in the singular is not intended to mean “one and only one” unless specifically so stated, but rather “one or more.”

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

As used herein, and unless the context dictates otherwise, the term “coupled to” is intended to include both direct coupling (in which two elements that are coupled to each other contact each other) and indirect coupling (in which at least one additional element is located between the two elements).

The recitation of ranges of values herein is merely intended to serve as a shorthand method of referring individually to each separate value falling within the range, and including the endpoints. Unless otherwise indicated herein, each individual value is incorporated into the specification as if it were individually recited herein.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “assembly,” “mechanism,” “element,” “device,” and the like may not be a substitute for the word “means.” As such, no claim element is to be construed as a means plus function unless the element is expressly recited using the phrase “means for.”

The accompanying claims and their equivalents are intended to cover such forms or modifications as would fall within the scope of the disclosure. The features and attributes of the specific example embodiments of the assemblies, apparatus, systems, and methods disclosed above may be combined in different ways to form additional embodiments, all of which fall within the scope of the disclosure.

1. A substance supply apparatus, comprising:
a base component comprising a plurality of walls and a cavity configured to house one or more electrical components;
a top plate affixed to the base component, the top plate enclosing the cavity and comprising a plurality of heating elements electrically coupled to the electrical components, wherein the plurality of heating elements includes a first heating element configured to receive a first end of a cartridge configured to house a substance to be heated; and
a manifold comprising a plurality of delivery ports including a first delivery port, and wherein the first delivery port is configured to couple with a second end of the cartridge and receive an inhalable drug produced in response to a heating of the substance by the first heating element.

2. The substance supply apparatus of claim 1, wherein the plurality of heating elements further comprises a second heating element configured to receive a first end of a second cartridge configured to house a second substance to be heated, and wherein the plurality of delivery ports comprises a second delivery port configured to couple with a second end of the second cartridge and receive a second inhalable drug produced in response to a heating of the second substance by the second heating element.

3. The substance supply apparatus of claim 2, wherein the plurality of heating elements further comprises a third heating element configured to receive a first end of a third cartridge configured to house a third substance to be heated, and wherein the plurality of delivery ports comprises a third delivery port configured to couple with a second end of the third cartridge and receive a third inhalable drug produced in response to a heating of the third substance by the third heating element.

4. The substance supply apparatus of claim 1, further comprising a controller configured to drive each of the heating elements of the plurality of heating elements separately and independently in accordance with a predefined order.

5. The substance supply apparatus of claim 3, wherein each of the first, second and third cartridges houses a different amount of the same substance.

6. The substance supply apparatus of claim 3, wherein each of the first, second and third cartridges houses a different type of substance.

7. The substance supply apparatus of claim 3, wherein each of the first, second and third cartridges houses a different concentration of the same substance.

8. The substance supply apparatus of claim 1, wherein the plurality of heating elements comprises ten heating elements arranged in a two by five array.

9. The substance supply apparatus of claim 1, wherein the top plate is circular, and wherein the plurality of heating elements comprises five heating elements.

10. A substance inhalation system, comprising:
an inhalation chamber having an inlet port having a fitting configured for releasable connection to a supply hose;
an actuator device;
a supply apparatus for supplying an inhalable substance to the inlet port, the supply apparatus comprising:
a base component comprising a plurality of walls and a cavity configured to house one or more electrical components;
a top plate affixed to the base component, the top plate enclosing the cavity and comprising a plurality of heating elements electrically coupled to the electrical components, wherein the plurality of heating elements includes a first heating element configured to receive a first end of a first cartridge configured to house a substance to be heated; and
a manifold comprising an outlet having a fitting for releasable connection to the supply hose, and a plurality of delivery ports including a first delivery port, wherein the first delivery port is configured to couple with a second end of the cartridge and receive an inhalable drug produced in response to a heating of the substance by the first heating element; and
a supply path comprising the supply hose between the supply apparatus and chamber inlet port; and
a controller connected to the supply apparatus and actuator device and configured to cause one or more heating elements of the plurality of heating elements to heat the substance housed in the cartridge.

11. The substance supply system of claim 10, wherein the plurality of heating elements further comprises a second heating element configured to receive a first end of a second cartridge configured to house a second substance to be heated, and wherein the plurality of delivery ports comprises a second delivery port configured to couple with a second end of the second cartridge and receive a second inhalable drug produced in response to a heating of the second substance by the second heating element.

12. The substance supply system of claim 11, wherein the plurality of heating elements further comprises a third heating element configured to receive a first end of a third cartridge configured to house a third substance to be heated, and wherein the plurality of delivery ports comprises a third delivery port configured to couple with a second end of the third cartridge and receive a third inhalable drug produced in response to a heating of the third substance by the third heating element.

13. The substance supply system of claim 10, wherein the controller is configured for user selection of a self-administration mode or passive mode, whereby the controller sequentially and separately turns on each heating element of the plurality of heating elements on separate and distinct activations of the actuator device by a test animal in the chamber when in a self-administration mode, and separately turns on each heating element of the plurality of heating elements in accordance with a predetermined order when in a passive mode.

14. The substance supply system of claim 12, wherein each of the first, second and third cartridges houses a different amount of the same substance.

15. The substance supply system of claim 12, wherein each of the first, second and third cartridges houses a different type of substance.

16. The substance supply system of claim 12, wherein each of the first, second and third cartridges houses a different concentration of the same substance.

17. The substance supply system of claim 10, wherein the plurality of heating elements comprises ten heating elements arranged in a two by five array.

18. The substance supply system of claim 10, wherein the top plate is circular, and wherein the plurality of heating elements comprises five heating elements.
